(19) United States
(12) Patent Application Publication
(10) Pub. No.: US 2009/0298852 A1
(43) Pub. Date: Dec. 3, 2009

(54) Pellet-form slow-release preparation for vertigo

(76) Inventor: Gernot Francas, Worms (DE)

Correspondence Address: KRIEGSMAN & KRIEGSMAN, 30 TURNPIKE ROAD, SUITE 9, SOUTHBOROUGH, MA, 01772, US

(21) Appl. No.: 11/887,025

(22) PCT Filed: Mar. 23, 2006
(86) PCT No.: PCT/DE2006/000546

§ 371 Date: May 15, 2009

(30) Foreign Application Priority Data
Mar. 23, 2005 (DE) 10-2005-014-142.0

(51) Int. Cl. A61K 31/4965; A61P 25/00

(52) U.S. Cl. 514/255.04

(57) ABSTRACT

The invention describes a pharmaceutical composition of a slow-release preparation in the form of pellets for the treatment of vertigo of any genesis. Thus, a pharmaceutical composition is described that contains cinnarizine and dimenhydrinate, wherein the release of active ingredients is slowed down and the preparation is in the form of pellets. In addition, the pharmaceutical composition contains binding agent, slow-release agent and fillers.

The invention relates to a pharmaceutical composition of a slow-release preparation in the form of pellets for the treatment of vertigo of any genesis.

Next to headache, vertigo or dizziness (Latin vertigo) is the most frequent symptom mentioned by patients. It involves a so-called multisensory syndrome, which is characterized by a disturbed sensation of different senses and is accompanied by the loss of the body's security in space and thus by equilibrium disturbances that are caused thereby. In full expression, vertigo is expressed by the sensation of seeing stars, a tendency to fall, as well as nausea and vomiting. Vertigo may occur both in episodes as well as continually.

Vertigo is understood as an unpleasant distortion of spatial and movement sensation, which leads to equilibrium disturbances. It does not involve a stand-alone disorder, but rather a complex of symptoms of different causes and origin, in which different body senses are involved.

A preparation for vertigo, which contains cinnarizine and dimenhydrinate in combination is known from DE 103 01 981 A1. It was surprisingly found that the combination of the active ingredients leads to a synergistic effect.

It is a disadvantage, however, that the effect subsides after a certain time and it is necessary to administer additional doses several times per day. Previously these drugs have had to be administered up to 5 times distributed throughout the day. The problems of compliance that go hand in hand with this are quite clear.

The object of the present invention is to provide a system for the treatment of vertigo, whose duration of action is prolonged when compared with conventional pharmaceuticals.

The object of the present invention is accomplished by a pharmaceutical composition according to the principal claim. Advantageous enhancements of the pharmaceutical composition according to the invention are characterized in the dependent subclaims.

The object is accomplished according to the invention by a pharmaceutical composition containing cinnarizine and dimenhydrinate, wherein the release of active ingredient is slowed down and the preparation is in the form of pellets. It is preferred according to the invention that this pharmaceutical composition additionally contains binding agent, slow-release agent and fillers.

It is further preferred that additional auxiliary agents are contained in it.

A pharmaceutical composition according to the invention is preferred, wherein the weight ratio of binding agent/filler in the core lies between 50/1 and 5/1 and the weight ratio of slow-release agent/additional auxiliary agents in the lacquer lies between 4/1 and 1.5/1.

A pharmaceutical composition according to the invention is most particularly preferred, wherein the weight ratio of binding agent/filler in the core lies between 33.12/1 and 6.25/1 and the weight ratio of slow-release agent/additional auxiliary agents in the lacquer lies between 3/1 and 2.2/1.

It is further preferred that the binding agent is selected from low-viscosity hydroxypropylmethylcellulose (HPMC)≦1000 cp, microcrystalline cellulose and/or polyethylene glycol (PEG).

It is also preferred that the slow-release agent is selected from low-viscosity hydroxypropylmethylcellulose (HPMC)≦1000 cp and/or Eudragit RL/RS/NE.

It is further particularly preferred that the slow-release agent is also a film forming agent.

Another subject of the present invention is the use of the pharmaceutical composition according to the invention for the treatment of vertigo of any genesis.

Another subject of the present invention is also the use of cinnarizine and dimenhydrinate or their physiologically compatible salts in combination for the treatment of vertigo of any genesis.

Cinnarizine (CAS 293-57-7) is the international free [nonproprietary] name (INN) for 1-benzhydryl-4-trans-cinnamylpiperazine. It involves an anti-vertigo preparation, which acts primarily as a calcium channel blocker on vestibular hair cells according to most recent knowledge. Dimenhydrinate (CAS 523-87-5) is the international free name (INN) for the 8-chlorotheophylline salt of diphenhydramine and is an antihistamine with anticholinergeic properties, which has anti-vertigo and anti-emetic actions. The solubilities of the active ingredients in water are very different, i.e., that of cinnarizine is approximately 2 mg/100 ml, while that of dimenhydrinate is approximately 3 mg/ml.

Because of the very different solubility behavior, it has previously not been possible to create a slow-release form that releases the active ingredients in such a timely manner that the synergistic effect of the combination of active ingredients is preserved.

It has now been found surprisingly that a combination of specific auxiliary agents provides the slow-releasing effect according to the invention, as long as they are present next to one another in a specific quantity ratio.

It could be shown that binding agent, slow-release agent and fillers must be present next to one another in a specific ratio in order to bring about the inventive effect of the timely release of the active ingredients.

The object of the invention is accomplished by a pharmaceutical composition which is described in the principal claim. Advantageous embodiments of the composition according to the invention are characterized in the dependent subclaims.

The object is accomplished by creating a slow-release composition, which contains binding agent, slow-release agent, and fillers in a defined weight ratio. This weight ratio according to the invention of binding agent:filler in the core of the pellets lies between 50:1 and 5:1, while the weight ratio of slow-release agent:additional auxiliary agents in the lacquer lies between 4:1 and 1.5:1.

It is particularly preferred that the weight ratio of binding agent:filler in the core of the pellets lies between 33.12:1 and 6.25:1, while the weight ratio of slow-release agent:additional auxiliary agents in the lacquer lies between 3:1 and 2.2:1.

It has been found surprisingly that with this quantity ratio, the release of the active ingredients dimenhydrinate and cinnarizine in optimal ratio to one another is achieved. The synergistic effect of the combination of these active ingredients can only be achieved in this way.

Pharmaceutical compositions according to the invention contain at least one binding agent, which is selected from low-viscosity hydroxypropylmethylcellulose (HPMC)≦1000 cp, hydroxypropylcellulose (HPC), microcrystalline cellulose and/or polyethylene glycol (PEG) or from their mixtures. Of course, other equivalent binding agents known to the person skilled in the art can also be used according to the invention. It is also possible to combine the named and other binding agents in order to obtain compositions according to the invention.

Pharmaceutical compositions according to the invention additionally contain at least one slow-release agent, which is selected from low-viscosity hydroxypropylmethylcellulose (HPMC)≦1000 cp, Eudragit RL, Eudragit RS and/or Eudragit NE or their mixtures. Of course, other equivalent slow-release agents known to the person skilled in the art can also be used according to the invention. It is also possible to combine the named and other slow-release agents in order to obtain compositions according to the invention.

Pharmaceutical compositions according to the invention additionally contain auxiliary agents such as softeners (e.g., triacetin, PEG and others), separating agents (e.g., talcum, glycerol monostearate and others), colorants and/or pigments or their mixtures. Such auxiliary agents are, for example, magnesium stearate, talcum, aerosil, separating agents, lubricants and the like, which facilitate and/or make possible the processing of the mixtures by machines. Of course, other equivalent auxiliary agents known to the person skilled in the art can also be used according to the invention. It is also possible to combine the named and other auxiliary agents in order to obtain compositions according to the invention.

Preparation forms that are suitable according to the invention are known to the person skilled in the art and may be pellets, capsules filled with pellets, e.g., of gelatin and other forms.

The preparation of such pharmaceutical forms is known to the person skilled in the art (e.g. Hagers Handbuch der Pharmazeut. Praxis [Hager's Handbook of Pharmaceutical Practice], 5th ed. of 1990-1995 of Springer Publishers, Berlin).

The following Examples explain the invention:

### EXAMPLE 1

### General Preparation Process for the Production of the Slow-release Preparations According to the Invention

The pharmaceutical compositions according to the invention are produced in a way known in and of itself. In this process,

dimenhydrinate, cinnarizine, binding agent and fillers are pre-mixed,
pelleted by means of water on a rotating disk, in a rotating drum or in the extruder, which classifies the pellets thus obtained into the particle size range of 600-1200 μm,
then the pellets are lacquer coated with a suspension of slow-release agent and the additional auxiliary agents in the presence of alcohol
and the lacquered pellets obtained in this way are introduced into gelatin capsules.

The compositions according to the invention were prepared according to different formulations as given above and their release was determined by the paddle method according to the European Pharmacopeia.

The invention will be explained in more detail in the following, based on the tables [and] figures. Taken individually

Table 2 shows a tabular presentation of the release of the pharmaceutical composition according to the invention according to formulation 1 of Table 1,

FIG. 1 shows a diagram of the release of cinnarizine,

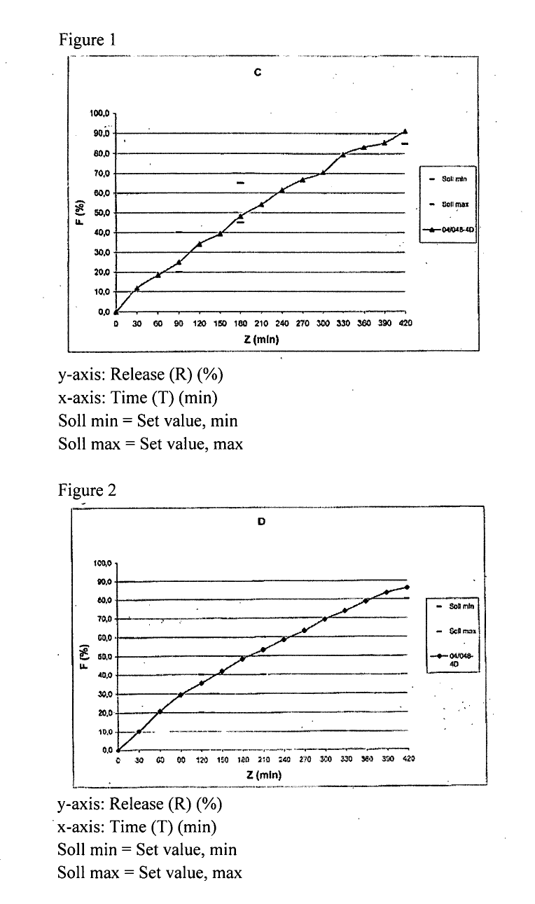

FIG. 2 shows a diagram of the release of dimenhydrinate,

Table 3 shows a tabular presentation of the release of a conventional cinnarizine/dimenhydrinate preparation,

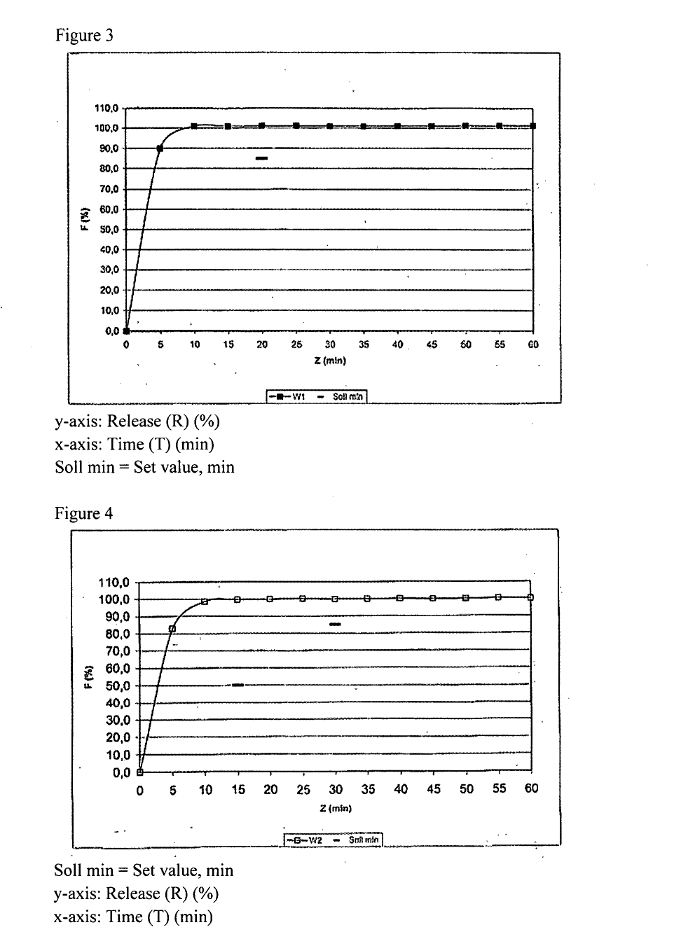

FIG. 3 shows a diagram of the release of cinnarizine and

FIG. 4 shows a diagram of the release of dimenhydrinate.

Table 2 shows a tabular presentation of the release of cinnarizine and dimenhydrinate with the use of the pharmaceutical composition according to the invention according to formulation 1 of Table 1.

A diagram of the release of cinnarizine (W1) from the composition according to the invention is shown in FIG. 1.

FIG. 2 shows a diagram in which the release (R) of dimenhydrinate (W2) is plotted as a function of time (T).

In comparison to Table 2, Table 3 gives a tabular presentation of the release of cinnarizine and dimenhydrinate for a conventional cinnarizine/dimenhydrinate preparation, not according to the invention.

FIG. 3 shows a diagram in which the release (R) of cinnarizine (W1) is plotted as a function of time (T).

FIG. 4 shows a diagram in which the release (R) of dimenhydrinate (W2) is plotted as a function of time (T).

The in-vitro release of cinnarizine base from the pharmaceutical composition according to the invention amounts to 20%-40% after 90 minutes, 45%-65% after 180 minutes and >85% after 420 minutes. The in-vitro release of dimenhydrinate amounts to 20%-40% after 120 minutes, 40%-60% after 210 minutes and >80% after 420 minutes.

A comparison of FIGS. 1 and 3 as well as FIGS. 2 and 4 shows that the release of cinnarizine or dimenhydrinate in the case of the unretarded mixture of cinnarizine and dimenhydrinate first increases very greatly and then remains at a value of approximately 100%, whereas the increase of release occurs in a delayed manner in the composition according to the invention.

### EXAMPLES 2-6

The following Table 1 shows formulations for the production of preparations according to the invention in the form of pellets.

TABLE 1

| | Formulations | | | | |
|---|---|---|---|---|---|
| Component | 1 | 2 | 3 | 4 | 5 |
| Active ingredient | | | | | |
| Dimenhydrinate | 120 | 120 | 120 | 120 | 120 |
| Cinnarizine | 60 | 60 | 60 | 60 | 60 |
| Binding agent | | | | | |
| Microcrystalline | 100 | 110 | 132 | 100 | 110 |
| cellulose | | | | | |
| HPMC of low viscosity | 20 | 20 | 7 | 20 | 20 |
| PEG | 5 | 5 | 1.75 | 5 | 5 |
| Filler | | | | | |
| Corn starch | 20 | 10 | 4.25 | 20 | 10 |

| | |
|---|---|
| Pellets - quantity | 325 mg |
| Pellets - size | 600-1200 μm |

Slow-release agent = Film-forming agent

| | | | | | |
|---|---|---|---|---|---|
| Eudragit RS | 10 | | | 10 | 10 |
| Eudragit RL | | | 10 | | |
| Eudragit NE | | 10 | | | |
| HPMC of low viscosity | 2 | 2 | 1 | 1 | 2 |
| Softener | | | | | |
| Triacetin | 2 | | 2 | 2 | 2 |
| PEG | | | | 1 | |
| Separating agent | | | | | |
| Talcum | 2 | 4 | | 2 | 1 |
| Glycerol monostearate | | | 3.7 | | |
| Colorants | | | | | |
| FeOx | | | 0.1 | | 0.5 |
| Pigments | | | | | |
| Titanium dioxide | | | 0.2 | | 0.5 |
| Film - quantity | 16 mg | 16 mg | 16 mg | 16 mg | 16 mg |

| | |
|---|---|
| Slow-release pellets - | 341 mg |
| quantity | |

The formulations according to the invention have the advantages of the invention, in that the active ingredients are released in a timely manner, so that their synergistic effect is maintained.

TABLE 2

| | Release Mean values (%) | | | | | |
|---|---|---|---|---|---|---|
| | Cinnarizine (C) | | | Dimenhydrinate (D) | | |
| Time | Set value | Set value | 04/048- | Set value | Set value | 04/048- |
| (min) | min | max | 4D | min | max | 4D |
| 0 | | | 0.0 | | | 0.0 |
| 30 | | | 11.8 | | | 10.2 |
| 60 | | | 18.5 | | | 20.7 |
| 90 | 20.0 | 40.0 | 25.0 | | | 29.4 |
| 120 | | | 34.2 | 20.0 | 40.0 | 35.4 |
| 150 | | | 39.5 | | | 41.8 |
| 180 | 45.0 | 65.0 | 48.1 | | | 48.5 |
| 210 | | | 54.1 | 40.0 | 60.0 | 53.2 |
| 240 | | | 61.5 | | | 58.5 |
| 270 | | | 66.8 | | | 63.2 |
| 300 | | | 70.5 | | | 69.2 |
| 330 | | | 79.6 | | | 73.6 |
| 360 | | | 83.3 | | | 78.6 |
| 390 | | | 85.6 | | | 83.2 |
| 420 | 85.0 | | 91.2 | 80.0 | | 85.9 |

TABLE 3

| Release Mean values (%) (R) | |
|---|---|
| Cinnarizine (W1) | Dimenhydrinate (W2) |

| Time (T) | Set value | | Set value | |
|---|---|---|---|---|
| (min) | min | 03/633 | min | 03/633 |
| 0 | | 0.0 | | 0.0 |
| 5 | | 89.9 | | 82.8 |
| 10 | | 100.9 | | 98.5 |
| 15 | | 100.8 | 50.0 | 99.5 |
| 20 | 85.0 | 100.9 | | 99.8 |
| 25 | | 101.0 | | 100.0 |
| 30 | | 100.9 | 85.0 | 99.7 |
| 35 | | 100.9 | | 99.8 |
| 40 | | 101.0 | | 100.0 |
| 45 | | 100.9 | | 99.7 |
| 50 | | 101.1 | | 99.9 |
| 55 | | 101.3 | | 100.3 |
| 60 | | 101.2 | | 100.1 |

1. A pharmaceutical composition containing cinnarizine und dimenhydrinate, hereby characterized in that the release of active ingredients is slowed down and the preparation is present in the form of pellets.

2. The pharmaceutical composition according to claim 1, further characterized in that it additionally contains binding agent, slow-release agent and fillers.

3. The pharmaceutical composition according to claim 1, further characterized in that additional auxiliary agents are contained in it.

4. The pharmaceutical composition according to claim 1, further characterized in that the weight ratio of binding agent/filler in the core lies between 50/1 and 5/1 and the weight ratio of slow-release agent/additional auxiliary agents in the lacquer lies between 4/1 and 1.5/1.

5. The pharmaceutical composition according to claim 3, further characterized in that the weight ratio of binding agent/filler in the core lies between 33.12/1 and 6.25/1 and the weight ratio of slow-release agent/additional auxiliary agents in the lacquer lies between 3/1 and 2.2/1.

6. The pharmaceutical composition according to claim 1, further characterized in that the binding agent is selected from low-viscosity hydroxypropylmethylcellulose (HPMC)≦1000 cp, microcrystalline cellulose and/or polyethylene glycol (PEG).

7. The pharmaceutical composition according to claim 1, further characterized in that the slow-release agent is selected from low-viscosity hydroxypropylmethylcellulose (HPMC)≦1000 cp and/or Eudragit RL/RS/NE.

8. The pharmaceutical composition according to claim 1, further characterized in that the slow-release agent is also a film forming agent.

9. The use of the pharmaceutical composition according to claim 1 for the treatment of vertigo of any genesis.
